(19) United States
(12) Patent Application Publication
(10) Pub. No.: US 2022/0160990 A1
(43) Pub. Date: May 26, 2022

(54) APPARATUS FOR DEFINING CPAP VENTILATION WITH A MINIMUM VOLUME

(71) Applicant: Loewenstein Medical Technology S.A., Luxembourg (LU)

(72) Inventors: Benjamin Adametz, Hamburg (DE); Marcel Mehnert, Kremperheide (DE)

(21) Appl. No.: 17/455,690

(22) Filed: Nov. 19, 2021

(30) Foreign Application Priority Data
Nov. 24, 2020 (DE) 102020007181.3

(51) Int. Cl. A61M 16/20; A61M 16/00; A61M 16/08; A61M 16/22

(52) U.S. Cl. CPC A61M 16/201; A61M 16/0003; A61M 16/0875; A61M 16/024; A61M 16/22; A61M 2016/0018; A61M 2016/0027; A61M 2016/0036; A61M 2230/42; A61M 2230/40; A61M 2205/52

(57) ABSTRACT

A ventilator for respiration gas supply, comprising a respiration gas source, a control unit, a memory, a pressure sensor and/or a flow sensor, an exchangeable respiration gas tube, at least one connection stub for the respiration gas tube, a patient interface and a valve. The control unit is set up to use signals from the pressure sensor and/or flow sensor to ascertain the patient's respiration phase and to ascertain the patient's current tidal volume during successive inhalations and exhalations and to compare a first set volume threshold for the tidal volume with the current tidal volume and to determine whether the latter is below the former and if so, to react by driving the respiration gas source to set a second pressure for the respiration gas for inhalation and driving the respiration gas source to set the CPAP pressure for the respiration gas for exhalation.

### CROSS-REFERENCE TO RELATED APPLICATIONS

The present application claims priority under 35 U.S.C. § 119 of German Patent Application No. 102020007181.3, filed Nov. 24, 2020, the entire disclosure of which is expressly incorporated by reference herein.

### BACKGROUND OF THE INVENTION

### 1. Field of the Invention

The present invention relates to an apparatus for defining a CPAP ventilation with a minimum volume.

### 2. Discussion of Background Information

Ventilators are used for the therapy of respiratory disorders; the ventilators may be used in non-invasive and invasive ventilation, either in a clinical setting or outside a clinical setting.

In the ventilation of a patient, it is generally possible to use a ventilator having an inhalation branch for inhalatory respiration gas flow and optionally having a branch for exhalatory respiration gas flow. The branch for exhalatory respiration gas flow enables the breathing-out/exhalation of a respiration gas by the patient, while the branch for inhalatory respiration gas supplies the patient with respiration gas.

In operation, the ventilators may be connected to a tube system with a passive exhalation opening/leakage tube system or a tube system with an active exhalation valve/valve tube system.

In ventilators known in the prior art, it is possible to switch between ventilation with a leakage tube system and a valve tube system. For this purpose, it has been necessary to date to undertake conversion/installation of one or more components externally or internally in the ventilator, for example of a nonreturn valve. A leakage tube system here is a one-tube system with a defined leakage opening through which respiration gas can escape constantly during ventilation in order to flush out carbon dioxide. A valve tube system is a one-tube system with a switchover valve for exhalation or a twin-tube system in which exhaled respiration gas is fed back to the ventilator for monitoring. Ventilators therefore have at least two connection stubs for either the twin-tube system or the one-tube system. In addition, ventilators have pressure stubs that are needed for the use of one-tube systems with a valve in order to guide a control pressure to the valve. In ventilators known in the prior art, therefore, it is always necessary to use an appropriate adapter for the selected tube system and, in addition, any pressure stubs will need to be opened or closed. Before the patient tube system can be connected, the appropriate tube system adapter must be installed. However, installation/conversion is time-consuming and prone to error and means a barrier to use. A further disadvantage is that the therapy mode is always dependent on the tube system. For instance, a CPAP mode always requires a leakage tube system for respiration gas to be able to escape constantly, in order to flush out carbon dioxide. A CPAP mode is currently not possible with a one-tube system having a switchover valve. In CPAP mode, spontaneous respiration by the patient is absolutely necessary for gas exchange. Breaks or a reduction in spontaneous respiration lead to reduced gas exchange and a risk to the patient.

In view of the foregoing, it would be advantageous to have available a ventilator that enables recognition of decreasing spontaneous respiration in CPAP mode and maintenance of medically necessary minimum ventilation for the patient, even in the case of decreasing spontaneous respiration.

It further would be advantageous to have available an apparatus that enables utilization of a ventilator both with a leakage tube system and with a valve tube system without conversion measures or without an adapter, and additionally to enable CPAP therapy with the same tube system.

### SUMMARY OF THE INVENTION

The present invention provides a ventilator as set forth in the independent claims. Developments and advantageous configurations are the subject of the dependent claims. Further advantages and features will be apparent from the general description and the description of the working examples.

The ventilator of the invention for respiration gas supply comprises a respiration gas source, a control unit, a memory, a pressure sensor device and/or a flow sensor device, an exchangeable respiration gas tube, at least one connection stub for the respiration gas tube and a patient interface, wherein the control unit drives the respiration gas source to set an essentially constant CPAP pressure which is maintained independently of the patient's respiration phase, and wherein the control unit is set up and designed to use the signals from the pressure sensor device and/or the flow sensor device
to ascertain the patient's respiration phase—inhalation and exhalation,
to ascertain the patient's current tidal volume during successive inhalations and exhalations,
to compare at least a first set volume threshold for the tidal volume with the current tidal volume,
to determine whether the current tidal volume is below the first volume threshold,
and if so to react by
driving the respiration gas source to set a second pressure (IPAP) for the respiration gas for inhalation
driving the respiration gas source to set the CPAP pressure for the respiration gas for exhalation.

Optionally or additionally, the ventilator is set up such that the control unit is set up and designed to increase the second pressure (IPAP) stepwise until the set volume threshold for the tidal volume has been attained.

Optionally or additionally, the ventilator is also set up such that the control unit increases the second pressure (IPAP) from one inhalation to the immediately subsequent inhalation.

The ventilator may also be set up such that the control unit lowers the second pressure (IPAP) stepwise again when the set volume threshold for the tidal volume has been exceeded.

It is also envisaged in accordance with the invention that the control unit lowers the second pressure (IPAP) to the CPAP pressure level when the set volume threshold for the tidal volume has been exceeded in a set manner and in this respect again drives the respiration gas source to set an essentially constant CPAP pressure which is maintained independently of the patient's respiration phase.

According to the invention, it is alternatively or additionally the case that the ventilator has at least one valve disposed in the respiration gas tube or in the ventilator.

According to the invention, the ventilator is set up such that the respiration gas tube in the event of a changeover from a CPAP mode to an IPAP mode remains on the ventilator, and the patient valve is switched by the control unit for IPAP mode.

The ventilator may also be set up such that the valve is opened or closed depending on the respiration phase.

The ventilator may additionally also be set up such that the valve is closed in the inhalation and is driven in a controlled manner in the exhalation, being opened intermittently in order to assure exhalation.

According to the invention, the ventilator is set up such that the patient's respiration is identified by the control unit from the progression of a flow signal from the flow sensor device, and the valve is actuated depending on the flow signal (as a trigger).

According to the invention, the ventilator is additionally set up such that limits are recorded or can be set for the flow signal and/or for a pressure signal, where the limits are the trigger sensitivity.

Optionally or additionally, the ventilator is set up such that the control unit drives the respiration gas source to assure maintenance of the CPAP pressure level during the switching operations of the valve.

Optionally or additionally, the ventilator is also set up such that the control unit at least intermittently lowers the CPAP pressure when the patient's respiration is identified as exhalation by the control unit from the progression of the flow signal from the flow sensor device.

Optionally or additionally, the ventilator is set up such that the control unit at least intermittently raises the CPAP pressure (pursed-lip breathing) when the patient's respiration is identified as exhalation by the control unit from the progression of the flow signal from the flow sensor device.

According to the invention, the ventilator is set up such that the control unit can set the CPAP pressure to pressure values below 4 hPa since, by virtue of the valve, CO₂ in the exhaled air is reliably flushed out even at low pressures.

Optionally or additionally, the ventilator is set up such that the control unit for CPAP mode keeps the valve closed in the inhalation and drives it in a controlled manner in the exhalation and opens it intermittently in order to assure exhalation, where the patient's respiration is identified by the control unit from the progression of the flow signal from the flow sensor device and the valve is actuated depending on the flow signal (as a trigger), where the maintenance of the CPAP pressure level is assured during the switching operations of the valve by driving of the respiration gas source.

According to the invention, it is also the case that a patient having difficulty in breathing (effortful inhalation) is identified by the control unit from the progression of the flow signal or of the pressure signal, and the control unit drives the respiration gas source at a set respiration gas flow or respiration gas pressure when the progression of the flow signal or of the pressure signal leads to identification of effortful inhalation by the patient. The invention also envisages that the pressure of the respiration assistance and the volume are adjustable.

Optionally or additionally, the ventilator of the invention is set up such that the pressure of the respiration assistance and an inhalation time Ti are adjustable.

Optionally or additionally, the ventilator is set up such that, for exhalation, the valve is opened briefly, such that the pressure is released, and the valve is then closed.

It is also a characteristic feature of the ventilator that the trigger sensitivity is adjustable in 3 to 15 stages.

It is an additional characteristic feature of the ventilator that a trigger block time (in the range of from 0.1 to 10 seconds) can be set, where the patient's respiration efforts are ignored by the control unit for the duration of the trigger block time.

Optionally or additionally, the ventilator is set up such that the control unit the control unit drives the respiration gas source to set a respiration gas pressure in the range of 0-90 mbar, preferably 1-80 mbar, more preferably 2-60 mbar.

Optionally or additionally, the ventilator is also set up such that it has a pressurized gas source and at least one pressure tube that guides a control pressure to the valve.

It is also a characteristic feature of the ventilator that the respiration gas source is the pressurized gas source.

Optionally or additionally, the ventilator is set up such that the respiration gas tube is a one-tube system with a valve.

Optionally or additionally, the ventilator is set up such that the respiration gas tube is a two-tube system with a valve.

According to the invention, the ventilator is set up such that the respiration gas tube is a two-tube system with an assigned valve, where the valve is adjacent to the stub in a ventilator housing.

Optionally or additionally, the ventilator is set up such that the patient valve is designed so as to be removable from a receptacle in the housing, where the patient valve has a membrane that may be subject to a control pressure in order to block or to clear a flow of respiration gas through the valve.

According to the invention, the ventilator is set up such that the valve has a sealing membrane which is subject to a control pressure that opens or closes the valve, where the control pressure is generated by the respiration gas source and is guided to the valve via a control tube.

The ventilator may also have a valve which is electrically operated.

According to the invention, the ventilator is set up such that the patient interface takes the form of a nasal cannula or flow cannula, of a nose plug or mask, or of a tracheostomy connector.

In a further development, the ventilator has a user interface set up and designed as an operating and display element. In general, the operating and display element takes the form of a graphical user interface (GUI). In general, the GUI takes the form of a touchscreen. Optionally, the operating and display element comprises tactile operating elements.

In one configuration, the ventilator comprises a digital interface set up to transmit detected parameters, measurements and information to a server or an external terminal and to receive data and information via the interface. The ventilator is optionally set up to store, to analyze and/or to assess the detected values and/or information from the measurement zone. The ventilator may be coupled via the interface to a cough assist device or another ventilator or a patient monitor and may exchange data.

The ventilator is optionally set up to transmit the measurements/parameters detected, analyzed and/or assessed to an external server. The transmission may be time-controlled, manually triggered (for example triggered on a home therapy device or on a server), event-controlled (for example in the event of recognition of particular critical states by the therapy device) or as a sustained transfer, at least during the course of therapy.

The measurements, parameters and information may be transferred every 2 hours to 7 days, especially every 1 to 3 days. In one execution, the transfer is effected at least once per day/per 24 hours. The interface may optionally be set up to transfer measurements, information or parameters hourly in collated form, or to transfer the measurements in real time. It is optionally possible for the user and/or a supervisor to freely choose a transfer cycle. The interface of the ventilator may be set up to conduct the transfer automatically, optionally in a repeated or sustained manner, after one or more fixedly programmed and/or freely input time intervals.

In the event of failure of a data connection, the memory unit of the apparatus may be set up to store the measurements and/or information for at least one day, in which case the interface of the apparatus is set up to transfer the data to an external server or a terminal as soon as a data connection has been restored.

The ventilator is optionally set up, via the operation and display element, to include information and/or values that are manually input by the user and/or by the supervisor in the evaluation of measurements.

In a further configuration, the ventilator includes an alarm unit with a loudspeaker set up to sound an alarm in the event of recognized events, in which case the ventilator comprises at least one microphone set up to monitor an alarm sounded by the alarm unit. This offers an additional safety function for the correct use of the ventilator apparatus.

In one configuration, the ventilator is set up to be combinable with other apparatuses. The ventilator optionally has a connection for a nebulizer, in which case the ventilator is set up to control a connected nebulizer via the ventilator. The ventilator is optionally set up to detect a return signal from the nebulizer and to take it into account in the control of the nebulizer.

The ventilator comprises connections for a server, a patient management system, a cough device and a sleep laboratory infrastructure. In addition, the ventilator comprises a cloud function, in which case the ventilator is set up to transmit data via an interface to a cloud or a connection for a GSM module. In one development, the ventilator comprises a connection for a nurse call module. In addition, the ventilator comprises at least an SpO₂ connection and/or a CO₂ connection.

The ventilator has the following operating states, for example:

On: Treatment is in progress. Ventilation and therapy settings are possible.

Standby: The fan is off and therapy is not in progress. However, the ventilator is ready for immediate operation. Ventilation and therapy settings are possible.

Off: The ventilator is switched off. No settings are possible and the display remains dark.

The ventilator is intended for constant or intermittent respiratory assistance for the care of persons in need of mechanical ventilation. The ventilator is also intended, for example, for children and adults with a minimum tidal volume of 30 ml. The ventilator is suitable for use in the domestic sector, in care facilities and hospitals, and for mobile applications, for example in a wheelchair or on a wheeled bed. It may be used for invasive and non-invasive ventilation. The ventilator is also intended for use as a ventilator during transport or in intensive care.

The ventilator may be used either with non-invasive or with invasive ventilation routes. A fan sucks in ambient air through a filter and conveys it at the therapy pressure via the ventilation tube and the ventilation route to the patient. On the basis of the signals detected from the pressure and flow sensors, the fan is controlled in accordance with the respiration phases. The user interface serves for display and adjustment of the parameters and alarms available. The ventilator may be used either with a leakage tube, a one-tube valve system or a twin-tube system. In the case of a leakage tube, an exhalation system is used to continuously flush out the CO₂-containing exhaled air. In the case of a one-tube valve system and in the case of a twin-tube system, the patient's exhalation is controlled by means of a valve.

An integrated FiO₂ sensor can be used if required to measure the FiO₂ concentration released by the ventilator. Feeding of an external SpO₂ measurement is also possible. Main supply takes place via an external power supply. The ventilator has an installed battery and can therefore continue to be operated without interruption in the event of a grid failure. In addition, it is possible for a maximum of two external batteries to be connected in order to operate the ventilator. Treatment data are stored in the ventilator and may additionally be loaded onto a USB-C stick and evaluated by means of PC software.

The respiration gas drive may be a fan, a valve, an oxygen source (high pressure) or an air pressure source (high pressure) or a combination of the above. The respiratory gas drive is arranged so as to be freely suspended in the ventilator, for example via at least two, especially three, suspension points.

The control unit generally comprises at least a memory unit and an evaluation unit. The memory unit is set up to store measurements, information and/or parameters and to provide them for evaluations by the evaluation unit. The evaluation unit is set up to compare the measurements, information and/or parameters with one another or with external data. The control unit is set up to receive data from components of the apparatus, especially a measurement unit of the flow measurement zone, and to store and analyze them. The control unit is optionally set up to transmit data, measurements, information and/or parameters to a digital interface of the apparatus.

The ventilator is especially also set up for use in pediatric ventilation. The apparatus includes recorded respiration modes. More particularly, the ventilator includes at least one high-flow mode and at least one PEEP control mode. In general, the control unit of the apparatus is set up to adjust the respiration modes, frequencies, triggers and flows of the apparatus.

The ventilator may be used with a leakage tube, a one-tube valve system or a twin-tube system. In the case of the leakage tube, an exhalation system is used to continuously flush out the CO₂-containing exhaled air.

In the one-tube valve system and in the twin-tube valve system, the patient's exhalation is controlled by means of a valve.

In the twin-tube system, the valve is disposed in the ventilator. The exhaled air is guided via a part-tube to the exhalation input stub of the ventilator and thence released into the environment via the valve. For this purpose, the valve opens with every exhalation. The valve is closed with every inhalation.

In the one-tube valve system, the valve is disposed in or on the tube. No matter whether internally or externally, the valve is always subject to a control pressure that opens or closes the valve.

The control pressure is generated by the respiration gas source and is guided via a control tube to the valve. The control pressure is guided first to the internal valve. The pressure can then be guided to the second valve (in the one-tube system). A blocker opens or blocks the way.

For the one-tube system, a pressure stub is disposed on the ventilator housing, to which the control pressure is applied. A pressure tube guides the control pressure to the valve.

The above-described invention may have the overall advantage that respiration of a patient is enabled, while patient mobility can be maintained. For example, the apparatus may be mounted on a wheelchair. The apparatus additionally includes, for example, a suction function and a cough mode. The ventilator may be adapted to various tube systems without conversion of the connection region of the tube system on the ventilator.

The inhalation time (Ti) may be adjusted for spontaneous respiration. In the range of 0.2 second to 4 seconds for children and 0.5 second to 5 seconds for adults.

Inhalation is ended no later than after Ti has elapsed.

Mandatory breath: Ti is fixed.

The trigger sensitivity can be adjusted in 10 stages. It is likewise possible to set a trigger block time. Inhalatory trigger signals are ignored within the set period, which is in the range of from 0.2 s to 5 s.

For the setting of volume: the inspired positive airway pressure (IPAP) can be set within the range of 4-50 hPa/mbar/cmH₂O when the leak tube system is used or within the range of 4-60 hPa/mbar/cmH₂O when the one-tube or twin-tube valve system is used.

The volume released (Vt) may be adjusted. In the range of from 30 ml to 400 ml for children and from 100 ml to 3000 ml for adults.

The trigger sensitivity can be adjusted in 10 stages. It is likewise possible to set a trigger block time. Inhalatory trigger signals are ignored within the set period, which is in the range of from 0.2 s to 5 s.

### BRIEF DESCRIPTION OF THE DRAWINGS

The invention is explained in greater detail below by way of exemplary embodiments with reference to the drawings, in which

FIG. 1A shows a ventilator according to the invention with a respiration mask as patient interface;

FIG. 1B shows different tube systems for the ventilator; and

FIG. 2 shows the setting for CPAP therapy.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

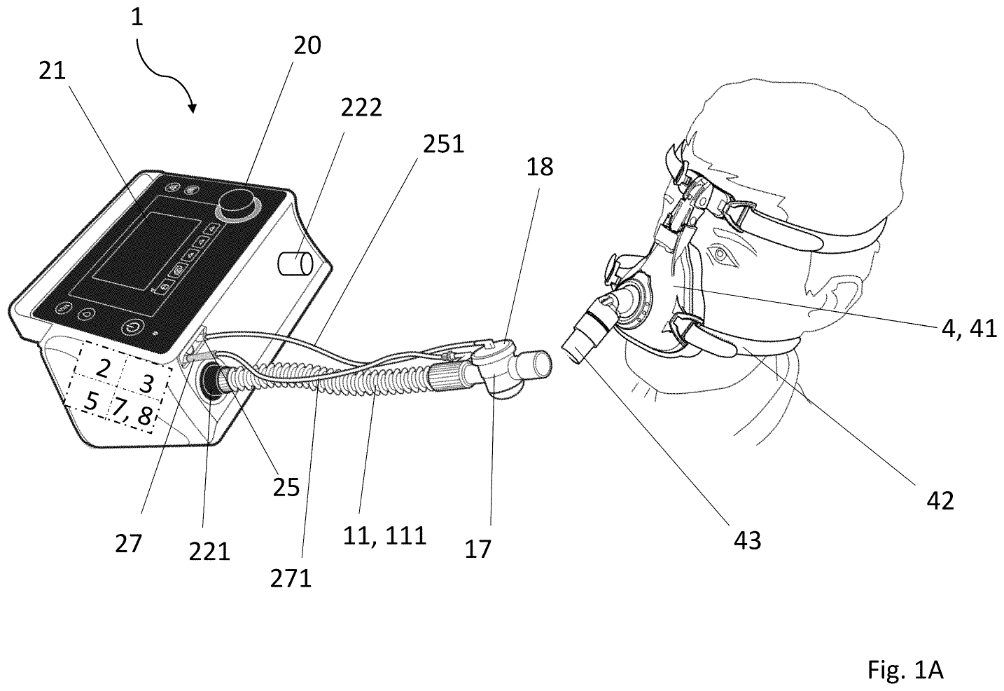

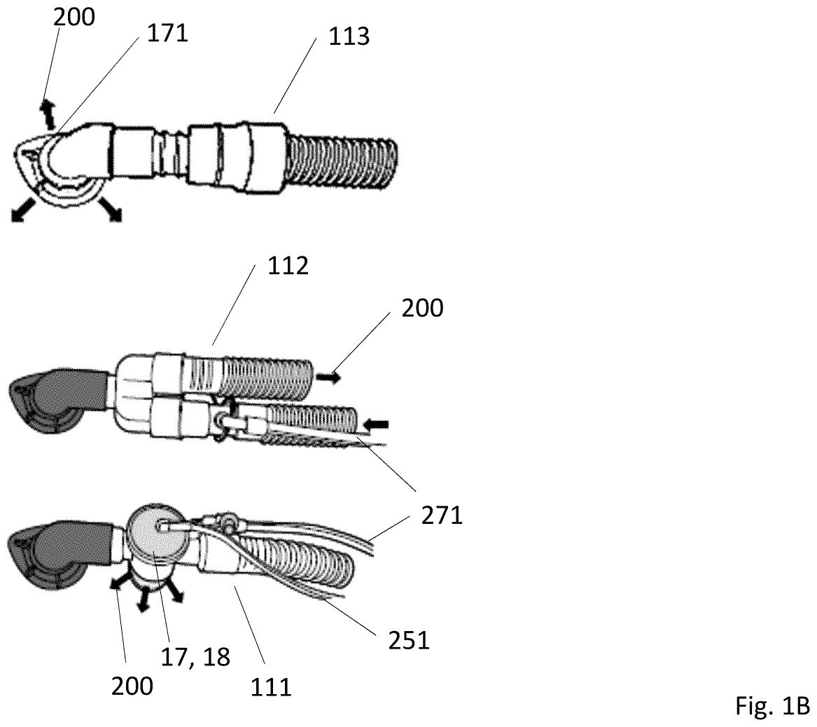

FIG. 1A shows a ventilator 1 according to the invention with a respiration mask 41 as patient interface 4. The mask is secured to the head with a strap 42. A stub 43 can be used to connect the mask to the tube.

The ventilator for respiratory gas supply 1 comprises a respiratory gas source 2, a control unit 3, a memory 5, a pressure sensor device 7 and/or a flow sensor device 8, a respiratory gas tube 11 and a patient interface 4, in the form here of a ventilation mask 41. The ventilator additionally has an operating unit 20 and a display 21. The ventilator additionally has two stubs 221, 222 for the respiratory gas tube 11. One stub 221 is set up for the connection of the respiratory gas tube 11 in the form of a one-tube valve system 111. A leakage tube 113 may also be connected to this stub 221 (see FIG. 1B).

In addition, the inhalatory branch of the twin-tube system 112 may be connected to this stub 221. The other stub 222 serves for connection of the exhalatory branch of the twin-tube system 112 (not shown).

The ventilator is set up and designed for respiration gas supply 1, comprising a respiration gas source 2, a control unit 3, a memory 5, a pressure sensor device 7 and/or a flow sensor device 8, an exchangeable respiration gas tube 11, at least one connection stub 221, 222 for the respiration gas tube and a patient interface 4, wherein the control unit 3 drives the respiration gas source 2 to set an essentially constant CPAP pressure which is maintained independently of the patient's respiration phase, and wherein the control unit 3 is set up and designed to use the signals from the pressure sensor device 7 and/or the flow sensor device 8
to ascertain the patient's respiration phase—inhalation and exhalation,
to ascertain the patient's current tidal volume during successive inhalations and exhalations,
to compare at least a first set volume threshold for the tidal volume with the current tidal volume,
to determine whether the current tidal volume is below the first volume threshold,
and if so to react by
   driving the respiration gas source 2 to set a second pressure (IPAP) for the respiration gas for inhalation
   driving the respiration gas source 2 to set the CPAP pressure for the respiration gas for exhalation.

FIG. 1B shows different tube systems. The ventilator may be used with a leakage tube 113 (top), a one-tube valve system 111 (bottom) or a twin-tube system 112 (middle).

In the leakage tube 113, an exhalation system 171 is used to continuously purge the CO2-containing exhaled air 200.

In the one-tube valve system and in the twin-tube valve system, the patient's exhalation is controlled via a valve 17.

In the twin-tube system 112, the valve 17 is disposed in the ventilator. The exhaled air is guided via a part-tube to the exhalation input stub 222 of the ventilator and thence released into the environment via the valve 17. For this purpose, the valve opens with every exhalation. The valve is closed with every inhalation. A pressure measurement tube 271 measures the pressure in the two-tube system.

In the one-tube valve system 111, the valve 17 is disposed in or on the tube 11.

The valve 17 has, for example, three fundamental gas pathways, and an opening provided with a sealing membrane. The gas pathways are a closable exhalatory gas pathway, an inhalatory gas pathway that points to the ventilator and through which inhalatory respiration gas flows, and a patient gas pathway that points to the patient interface. Inhalatory respiration gas flows through the patient gas pathway in inhalation, and exhalatory respiration gas in exhalation. The exhalatory gas pathway communicates with the opening that can be completely closed or opened by means of the membrane.

The opening and sealing membrane are covered in FIG. 1A by a closure 18. The pressure tube 251 opens at the closure 18. The pressure tube 251 guides the control pressure to the membrane. The membrane then closes the opening that leads to the exhalatory gas pathway.

The valve may be pneumatically operated and/or controlled. No matter whether it is disposed in the ventilator or on the tube, the valve is subject, for example, to a control pressure that opens or closes the valve. The valve has a sealing membrane which is subject to a control pressure that opens or closes the valve, with the control pressure generated by the respiration gas source 2 and guided through a control tube (not shown) to the valve 17.

A pressure measurement tube 271 measures the pressure in the tube adjacent to the valve. The control pressure is generated by the respiration gas source 2 and is guided to the valve via a control tube (not shown). The control pressure here is guided first, for example, to the internal valve 17 disposed adjacent to the exhalation input stub 222. The pressure can also be guided thence to the second valve 17 (in the one-tube system). A blocker (not shown) opens or blocks the pathway to the one-tube valve system 111.

For the one-tube system, a pressure stub 25 is disposed on the ventilator housing, to which the control pressure is applied. A pressure tube 251 guides the control pressure to the valve 17. The pressure stub 25 may be closed in order to prevent a pressure drop when the system is not in use.

Adjacent to the stub 221 is also disposed a stub 27 for a measurement of pressure. Pneumatically assigned to the stub 27 in the respirator is a pressure sensor. A pressure measurement tube 271 may be adapted to the stub 27, and this determines the pressure in the tube in the region (in flow direction) upstream of or beyond the valve 17.

Adjacent to the stub 222 may also be disposed a stub for a manometer. Pneumatically assigned to the stub in the respirator is a pressure sensor. A pressure measurement tube 271 may be adapted to the stub, which determines the pressure in the exhalatory tube or in the region (in flow direction) upstream of or beyond the valve. This measurement of pressure is advisable in order to determine and optionally to control compliance with the set pressure for the exhalation.

According to the invention, the valve 17 may be electronically controlled. In that case, it is supplied with energy, for example, via a cable connection from the ventilator or via a battery disposed adjacent to the valve.

Alternatively, the valve may be electrically operated and/or controlled; for example, the membrane is then moved against the opening by means of an electrically operated actuator.

Alternatively, the valve may be operated and/or controlled electrically, for example as an axial voice coil actuator. These consist of a permanent magnet in a movable tubular coil made of wire present in a ferromagnetic cylinder. When current flows through the coil, it becomes magnetized and repels the magnets. In this way, movement inward and outward and back and forth is generated. Further advantages of linear VCA motors are their bidirectionality and the presence of permanent magnets and magnetic hold-on coils. They make it possible to remain at one end of the stroke when the power supply is interrupted—in order to ensure, for example, that valves in the event of power failure remain open or closed. VCAs are accelerated uniformly and rapidly within the stroke, virtually without hysteresis.

The valve in the tube and/or the valve in the ventilator may be electrically controlled.

In this configuration, the ventilator is set up, for example, for CPAP mode 61.

According to the user's selection or in an automatically activated manner, the control unit 3 activates the CPAP therapy mode 61. The respiration gas source 2 is driven here to set a constant CPAP pressure. The CPAP pressure is preferably maintained independently of the respiration phase.

In the changeover to CPAP mode 61, the respiration gas tube 11 remains on the ventilator 1. The patient valve 17 is switched over here by the control unit for the setting of CPAP mode 61.

The valve 17 is closed in inhalation and driven in a controlled manner in exhalation and intermittently opened in order to assure breathing-out.

For this purpose, the patient respiration is identified by the control unit 3 from the progression of the flow signal of the flow sensor device 8, and the valve 17 is actuated depending on the flow signal (as trigger).

Limits are recorded or can be set for the flow signal. The limits represent the changeover between inhalation and exhalation, and hence serve as trigger signals for the driving of the valve 17. According to the invention, a pressure trigger is also possible, or a combination of the two trigger options. In the case of a pressure trigger, inhalation is recognized by the control unit in that the pressure drops slightly, and exhalation is recognized in that the pressure rises slightly. Limits are recorded or can be set for the pressure signal. The limits represent the changeover between inhalation and exhalation, and hence serve as trigger signals for the driving of the valve 17.

The control unit 3 controls the respiration gas source, for assurance of the maintenance of the CPAP pressure level, during the switchover operations of the valve 17.

The control unit 3 lowers the CPAP pressure, for example at least intermittently, when the patient's breathing is identified as exhalation by the control unit 3 from the progression of the flow signal of the flow sensor device 8. This makes it more pleasant for the patient to breathe out.

The control unit 3 raises the CPAP pressure alternatively, for example at least intermittently (pursed-lip breathing), when the patient's breathing is identified as exhalation by the control unit 3 from the progression of the flow signal of the flow sensor device 8. The elevated pressure allows closed regions of the lung to be opened up;

complete exhalation is then possible.

The control unit 3 can also set the CPAP pressure to pressure limits below 4 hPa since, by virtue of valve 17, in accordance with the degree of opening of the valve, CO2 in the exhaled air is reliably flushed out even at low pressures.

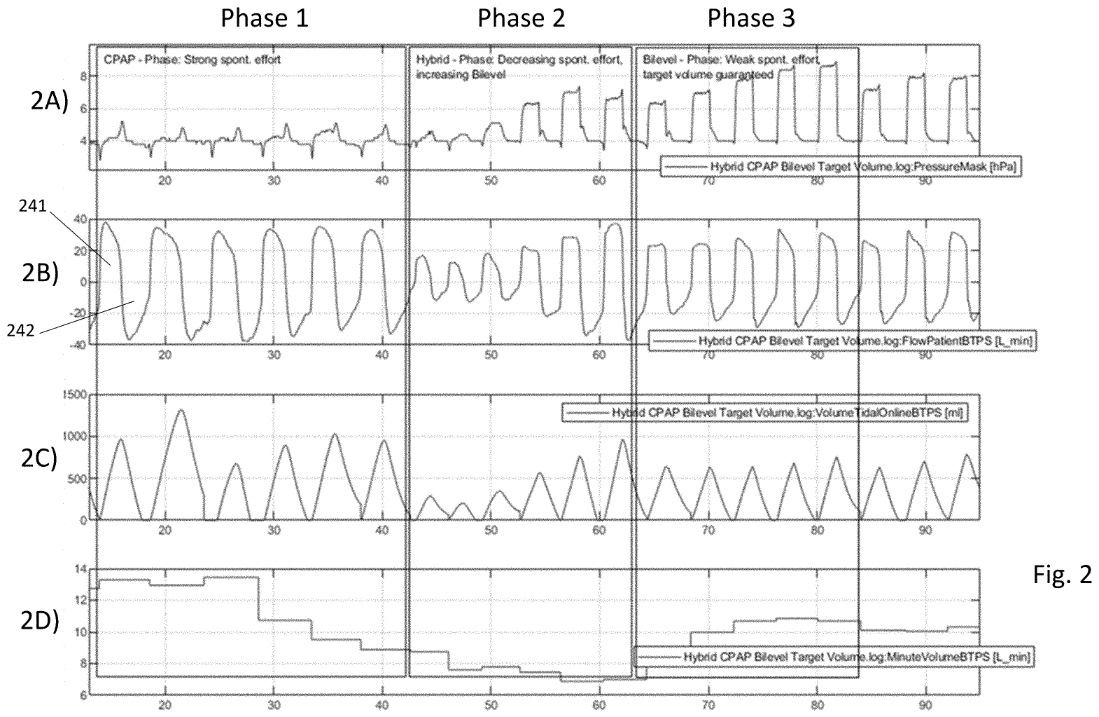

FIG. 2 shows the setting for CPAP therapy.

In FIG. 2A), pressure is shown on the Y axis. What is plotted is the mask pressure 32 as determined by the pressure sensor 7 (although the target pressure 33 that is set as the set value of the control unit 3 for the respiration gas source at least approximately corresponds to the mass pressure at least in phases). The pressure is reported in the unit hPa.

In phase 1, the CPAP pressure (of 4 hPa) is applied, and the patient breathes spontaneously. In phase 1, it is apparent that the CPAP pressure on the mask varies slightly at the changeover from inhalation 241 to exhalation 242 (see FIG. 2B). With every commencement of inhalation 241, the pressure drops below the target pressure since the patient develops a negative suction with their spontaneous breathing that the pressure regulator is unable to respond to as quickly.

With every commencement of exhalation 242, the pressure rises above the target pressure since the patient develops a positive flow (into the mass) with their spontaneous breathing that the pressure regulator is unable to respond to as quickly.

FIG. 2B) shows, on the Y axis, the flow rate (in L per minute), from which the patient's respiration phase 24 can be recognized. Respiration phase 24 is composed of at least one inhalation 241 and/or one exhalation 242.

It becomes clear from a comparison of FIG. 2B) and FIG. 2A) that the changeover from inhalation 241 to exhalation 242 regularly takes place in the rhythm of the patient's spontaneous breathing. An inhalation 241 corresponds to positive flows. An exhalation 242 corresponds to negative flows.

In FIG. 2C), the volume in ml is plotted on the Y axis. It is apparent that the volume in phase 1 in the inhalation 241 is always above 500 mL. In phase 2, the volume falls below 500 mL. A volume of 500 mL is set here by way of example as limit. Since 500 mL has been set as the limit below which the volume must not fall in CPAP therapy, the inventive assistance of respiration is activated by the control unit. It is apparent that the volume is rising gradually. The inventive assistance of respiration increases the CPAP pressure for inhalation and leaves the CPAP pressure unchanged for exhalation (or lowers the CPAP pressure for exhalation). The inventive assistance of respiration increases the CPAP pressure for inhalation stepwise, as apparent from FIG. 2A) in phase 2 and phase 3. As a result, there is an increase in the flow rate and volume again (cf. FIGS. 2B), 2C) and 2D)).

Plotted in FIG. 2D) is the minute volume in L per minute. It is apparent that the minute volume falls in phase 1 and assumes values below 8 L/min in phase 2. Since, alternatively or additionally to the volume according to FIG. 2C, a minute volume of 8 L/min may also be set here as the limit below which the minute volume must not fall in CPAP therapy, the inventive assistance of breathing is activated by the control unit. It is apparent that the minute volume is raised gradually in phase 3.

It is apparent from the progression of the mask pressure in FIG. 2A) with the flow rate from FIG. 2B) that the brief pressure drop corresponds to the patient's spontaneous inhalation 241. The pressure drop correlates in time with the rise in patient flow rate in FIG. 2B). This respiration signal from the patient can be regarded as a trigger by the control system in order to increase the control pressure specifically for inhalation. When the volume goes below the target volume (tidal volume or minute volume) or when the oxygen saturation SpO₂ goes below the target or when the etCO₂ value (final exhalatory CO₂ value in the respiration gas) goes below the target, there is a changeover to an IPAP pressure greater than the CPAP set; the CPAP becomes the exhalatory pressure (EPAP pressure).

The current pressure differential from the previous breath (CPAP or varying IPAP level) is apparent
a. from a table that assigns a pressure increase to the differential (target to actual volume or target to actual SpO₂ or target to actual CO₂) (stored in the memory)
b. from a value set by a user that assigns a pressure increase to the differential (target to actual volume or target to actual SpO₂ or target to actual CO₂)
c. from a combination of a) and b)
d. from a (self-taught) algorithm with the aid of estimating the (patho)physiological response to an increase in pressure, taking account of all input parameters, pressure, flow, volume, frequency, SpO₂, etCO₂.

In the case of exceedance of the target volume (tidal volume or minute volume) or in the case of exceedance of the target oxygen saturation SpO₂ or in the case that the etCO₂ value (final exhalatory CO₂ value) in the respiration gas goes below the target, there is a changeover to an IPAP pressure lower than the previously set IPAP (the CPAP value remains the EPAP), or a changeover back to the CPAP pressure.

The current pressure differential from the IPAP of the previous breath is apparent
a. from a table that assigns a pressure decrease to the differential (target to actual volume or target to actual SpO₂ or target to actual CO₂) (stored in the memory)
b. from a value set by a user that assigns a pressure decrease to the differential (target to actual volume or target to actual SpO₂ or target to actual CO₂)
c. from a combination of a) and b)
d. from a (self-taught) algorithm with the aid of estimating the (patho)physiological response to a decrease in pressure, taking account of all input parameters, pressure, flow, volume, frequency, SpO₂, etCO₂).
To sum up, the present invention provides:

1. A ventilator for respiration gas supply, wherein the ventilator comprises a respiration gas source, a control unit, a memory, a pressure sensor device and/or a flow sensor device, an exchangeable respiration gas tube, at least one connection stub for the respiration gas tube and a patient interface, wherein the control unit drives the respiration gas source to set an essentially constant CPAP pressure which is maintained independently of a patient's respiration phase, and wherein the control unit is set up and configured to use signals from the pressure sensor device and/or the flow sensor device
to ascertain the patient's respiration phase—inhalation and exhalation,
to ascertain the patient's current tidal volume during successive inhalations and exhalations,
to compare at least a first set volume threshold for a tidal volume with a current tidal volume,
to determine whether the current tidal volume is below the first set volume threshold,
and if so to react by
   driving the respiration gas source to set a second pressure (IPAP) for a respiration gas for inhalation
   driving the respiration gas source to set the CPAP pressure for a respiration gas for exhalation.

2. The ventilator of item 1, wherein the control unit is set up and configured to increase the second pressure (IPAP) stepwise until the set volume threshold for the tidal volume has been attained.

3. The ventilator of item 1 or item 2, wherein the control unit increases the second pressure (IPAP) from one inhalation to an immediately subsequent inhalation.

4. The ventilator of any one of the preceding items, wherein the control unit lowers the second pressure (IPAP) stepwise again when the set volume threshold for the tidal volume has been exceeded.

5. The ventilator of any one of the preceding items, wherein the control unit lowers the second pressure (IPAP) to the CPAP pressure level when the set volume threshold for the tidal volume has been exceeded in a set manner and in this respect again drives the respiration gas source to set an essentially constant CPAP pressure which is maintained independently of the patient's respiration phase.

6. The ventilator of any one of the preceding items, wherein the ventilator has at least one valve disposed in a respiration gas tube or in the ventilator.

7. The ventilator of any one of the preceding items, wherein the respiration gas tube in the event of a changeover from a CPAP mode to an IPAP mode remains on the ventilator, and the patient valve is switched by the control unit for IPAP mode.

8. The ventilator of item 6, wherein the valve is opened or closed depending on the respiration phase.

9. The ventilator of item 6, wherein the valve is closed in an inhalation and is driven in a controlled manner in an exhalation, being opened intermittently in order to assure exhalation.

10. The ventilator of item 6, wherein the patient's respiration is identified by the control unit from a progression of a flow signal from the flow sensor device, and the valve is actuated depending on the flow signal (as a trigger).

11. The ventilator of item 10, wherein limits are recorded or can be set for the flow signal and/or for a pressure signal, where the limits are the trigger sensitivity.

12. The ventilator of item 6, wherein the control unit drives the respiration gas source to assure maintenance of the CPAP pressure level during switching operations of the valve.

13. The ventilator of any one of the preceding items, wherein the control unit at least intermittently lowers the CPAP pressure when the patient's respiration is identified as exhalation by the control unit from a progression of the flow signal from the flow sensor device.

14. The ventilator of any one of the preceding items, wherein the control unit at least intermittently raises the CPAP pressure (pursed-lip breathing) when the patient's respiration is identified as exhalation by the control unit from a progression of the flow signal from the flow sensor device.

15. The ventilator of item 6, wherein the control unit can set the CPAP pressure to pressure values below 4 hPa since, by virtue of the valve, CO₂ in exhaled air is reliably flushed out even at low pressures.

16. The ventilator of item 6, wherein the control unit for CPAP mode keeps the valve closed in an inhalation and drives it in a controlled manner in an exhalation and opens it intermittently in order to assure exhalation, where the patient's respiration is identified by the control unit from a progression of the flow signal from the flow sensor device and the valve is actuated depending on the flow signal (as a trigger), where a maintenance of the CPAP pressure level is assured during switching operations of the valve by driving of the respiration gas source.

17. The ventilator of any one of the preceding items, wherein a patient having difficulty in breathing (effortful inhalation) is identified by the control unit from a progression of the flow signal or of the pressure signal, and the control unit drives the respiration gas source at a set respiration gas flow or respiration gas pressure when a progression of the flow signal or of the pressure signal leads to identification of effortful inhalation by the patient.

18. The ventilator of any one of the preceding items, wherein a pressure of a respiration assistance and a volume are adjustable.

19. The ventilator of any one of the preceding items, wherein a pressure of a respiration assistance and an inhalation time Ti are adjustable.

20. The ventilator of item 6, wherein, for exhalation, the valve is opened briefly, such that pressure is released, and the valve is then closed.

21. The ventilator of any one of the preceding items, wherein a trigger sensitivity is adjustable in 3 to 15 stages.

22. The ventilator of any one of the preceding items, wherein a trigger block time (in a range of 0.1 to 10 seconds) can be set, where the patient's respiration efforts are ignored by the control unit for a duration of the trigger block time.

23. The ventilator of any one of the preceding items, wherein the control unit drives the respiration gas source to set a respiration gas pressure in a range of 0-90 mbar, preferably 1-80 mbar, more preferably 2-60 mbar.

24. The ventilator of item 6, wherein the ventilator comprises a pressurized gas source and at least one pressure tube that guides a control pressure to the valve.

25. The ventilator of item 24, wherein the respiration gas source is the pressurized gas source.

26. The ventilator of any one of the preceding items, wherein the respiration gas tube is a one-tube system with valve.

27. The ventilator of any one of the preceding items, wherein the respiration gas tube is a two-tube system with valve.

28. The ventilator of any one of the preceding items, wherein the respiration gas tube is a two-tube system with an assigned valve, the valve being adjacent to a stub in a ventilator housing.

29. The ventilator of any one of the preceding items, wherein a patient valve is designed so as to be removable from a receptacle in a housing, where the patient valve comprises a membrane that may be subject to a control pressure in order to block or to clear a flow of respiration gas through the valve.

30. The ventilator of item 6, wherein the valve has a sealing membrane which is subject to a control pressure that opens or closes the valve, where the control pressure is generated by the respiration gas source and is guided to the valve via a control tube.

31. The ventilator of item 6, wherein the valve is electrically operated.

32. The ventilator of any one of the preceding items, wherein the patient interface is present in the form of a nasal cannula or flow cannula, of a nose plug or mask, or of a tracheostomy connector.

What is claimed is:

1. A ventilator for respiration gas supply, wherein the ventilator comprises a respiration gas source, a control unit, a memory, a pressure sensor device and/or a flow sensor device, an exchangeable respiration gas tube, at least one connection stub for the respiration gas tube and a patient interface, wherein the control unit drives the respiration gas source to set an essentially constant CPAP pressure which is maintained independently of a patient's respiration phase, and wherein the control unit is set up and configured to use signals from the pressure sensor device and/or the flow sensor device
to ascertain the patient's respiration phase—inhalation and exhalation,
to ascertain the patient's current tidal volume during successive inhalations and exhalations,
to compare at least a first set volume threshold for a tidal volume with a current tidal volume,
to determine whether the current tidal volume is below the first set volume threshold,
and if so to react by
driving the respiration gas source to set a second pressure (IPAP) for a respiration gas for inhalation
driving the respiration gas source to set the CPAP pressure for a respiration gas for exhalation.

2. The ventilator of claim 1, wherein the control unit is set up and configured to increase the second pressure (IPAP) stepwise until the set volume threshold for the tidal volume has been attained.

3. The ventilator of claim 2, wherein the control unit increases the second pressure (IPAP) from one inhalation to an immediately subsequent inhalation.

4. The ventilator of claim 3, wherein the control unit lowers the second pressure (IPAP) stepwise again when the set volume threshold for the tidal volume has been exceeded.

5. The ventilator of claim 1, wherein the control unit lowers the second pressure (IPAP) to the CPAP pressure level when the set volume threshold for the tidal volume has been exceeded in a set manner and in this respect again drives the respiration gas source to set an essentially constant CPAP pressure which is maintained independently of the patient's respiration phase.

6. The ventilator of claim 1, wherein the ventilator has at least one valve disposed in a respiration gas tube or in the ventilator.

7. The ventilator of claim 6, wherein the respiration gas tube in the event of a changeover from a CPAP mode to an IPAP mode remains on the ventilator, and the patient valve is switched by the control unit for IPAP mode.

8. The ventilator of claim 6, wherein the valve is opened or closed depending on the respiration phase.

9. The ventilator of claim 6, wherein the valve is closed in an inhalation and is driven in a controlled manner in an exhalation, being opened intermittently to assure exhalation.

10. The ventilator of claim 6, wherein the patient's respiration is identified by the control unit from a progression of a flow signal from the flow sensor device, and the valve is actuated depending on the flow signal (as a trigger).

11. The ventilator of claim 10, wherein limits are recorded or can be set for the flow signal and/or for a pressure signal, where the limits are the trigger sensitivity.

12. The ventilator of claim 6, wherein the control unit drives the respiration gas source to assure maintenance of the CPAP pressure level during switching operations of the valve.

13. The ventilator of claim 1, wherein the control unit at least intermittently lowers the CPAP pressure when the patient's respiration is identified as exhalation by the control unit from a progression of the flow signal from the flow sensor device.

14. The ventilator of claim 1, wherein the control unit at least intermittently raises the CPAP pressure (pursed-lip breathing) when the patient's respiration is identified as exhalation by the control unit from a progression of the flow signal from the flow sensor device.

15. The ventilator of claim 6, wherein the control unit can set the CPAP pressure to pressure values below 4 hPa since, by virtue of the valve, CO₂ in exhaled air is reliably flushed out even at low pressures.

16. The ventilator of claim 6, wherein the control unit for CPAP mode keeps the valve closed in an inhalation and drives it in a controlled manner in an exhalation and opens it intermittently in order to assure exhalation, where the patient's respiration is identified by the control unit from a progression of the flow signal from the flow sensor device and the valve is actuated depending on the flow signal (as a trigger), where a maintenance of the CPAP pressure level is assured during switching operations of the valve by driving of the respiration gas source.

17. The ventilator of claim 1, wherein a patient having difficulty in breathing (effortful inhalation) is identified by the control unit from a progression of the flow signal or of the pressure signal, and the control unit drives the respiration gas source at a set respiration gas flow or respiration gas pressure when a progression of the flow signal or of the pressure signal leads to identification of effortful inhalation by the patient.

18. The ventilator of claim 1, wherein a pressure of a respiration assistance and a volume are adjustable.

19. The ventilator of claim 1, wherein a pressure of a respiration assistance and an inhalation time Ti are adjustable.

20. The ventilator of claim 6, wherein, for exhalation, the valve is opened briefly, such that pressure is released, and the valve is then closed.
